(19) United States
(12) Patent Application Publication
(10) Pub. No.: US 2023/0026367 A1
(43) Pub. Date: Jan. 26, 2023

(54) SYSTEM AND METHOD FOR RENTING MEDICAL DEVICES

(71) Applicant: Zlasers Ltd., (US)

(72) Inventor: Moshe Ein-Gal, Ramat Hasharon (IL)

(21) Appl. No.: 17/382,451

(22) Filed: Jul. 22, 2021

(51) Int. Cl. G16H 40/40; G16H 20/00; G06Q 30/06

(52) U.S. Cl. CPC G16H 40/40; G16H 20/00; G06Q 30/0645

(57) ABSTRACT

A method of treatment includes providing one or more medical-device rental outlets, each of the outlets including one or more trained operators and one or more energy-emitting medical devices to perform a multiplicity of sequentially-performed energy-emitting self-treatment sessions by a patient. The sessions take place at the one or more medical-device rental outlets or at close proximity thereto. The patient pays a rental fee, generally related to the duration of each treatment session, rather than to the whole treatment duration.

### FIELD OF THE INVENTION

The present invention generally relates to renting or leasing of medical devices to improve medical care for the public without problems of home use.

### BACKGROUND OF THE INVENTION

Japanese Patent Application JP2002276177 describes a composite medical facility in which medical doctors can operate clinics in accordance with their medical treatment policies and management policies The composite medical facility has a plurality of clinics, which are respectively operated by different management subjects and, at the same time, can be leased, including various types of medical appliances installed in the clinics. In addition, the clinics are adjustable, that is, they can be changed in layout and the medical appliances which are used or leased can be changed to meet the demands of the lessor and lessees. The medical facility has an integrated receiving and accounting room, exclusively used inspection clinic, exclusively used medical treatment room, restaurant for outsiders, checkroom, and other commonly used facilities.

However, that patent application involves setting up many clinics, which are subject to medical and legal regulations, and the whole process and system becomes complicated and expensive.

In addition, medical devices for home use, such as wheelchairs, may be rented by users, i.e., care recipients. The rental period is typically long (e.g., weeks or months) and the device is used at the home of the care recipient. Multi-sessions and low risk treatments, such as treatment of erectile dysfunction by low intensity shockwaves, involve short sessions of less than one hour each. Due to the number of sessions and the time between successive sessions, the total overall treatment time is on the order of weeks or months. Electromedical devices for such multi-sessions treatments are more expensive than typical devices for home use, and rental of such devices for the duration of several weeks or months may be prohibitively expensive. Consequently, while such devices for multi-sessions treatments may be configured for home treatment, rental for such a long period is currently not implemented by care recipients. Thus, a need exists for a solution to the distribution problem of how to provide healthcare professionals with resources allowing the professional to perform medical treatments with minimum upfront investment and a need also exists for care recipients to afford renting devices for multi-sessions treatments.

### SUMMARY OF THE INVENTION

The present invention seeks to provide medical device rental outlets (as opposed to medical clinics) for renting electro-medical devices intended for multi-sessions treatments, wherein the rental period is short (for example, generally sufficient for conducting a session), and the session takes place at or close to the outlet. As opposed to medical clinics, such outlets are merely rental outlets, and as such, are subject to less regulations, are easier to manage, and are less expensive to set up and run. Consequently, while rental customers are subject to local and federal regulations related to the treatments, the rental fee for the devices may be low.

The terms renting and leasing are used interchangeably throughout.

There is provided in accordance with a non-limiting embodiment of the invention a method of treatment including providing one or more medical-device rental outlets, each of the outlets including one or more trained operators and one or more energy-emitting medical devices allowing a care recipient to rent an energy-emitting device and perform, assisted when needed by the trained operator, a multiplicity of sequentially-performed energy-emitting treatment sessions which take place at the one or more medical-device rental outlets or at close proximity thereto. The rental period of the energy-emitting device may be the time period for a full session or may be for other periods of time.

In accordance with a non-limiting embodiment of the invention, the sequentially-performed energy-emitting treatment sessions are performed in more than one of the outlets.

In accordance with a non-limiting embodiment of the invention, the sequentially-performed energy-emitting treatment sessions include shared usage of the one or more energy-emitting medical devices for treatment of more than one patient.

In accordance with a non-limiting embodiment of the invention, the sequentially-performed energy-emitting treatment sessions are performed using a low-intensity shockwaves device, such as but not limited to, for use in sexual wellness improvement, treatment of erectile dysfunction, treatment of a diabetic ulcer, vaginal treatments, pain and cosmetic indications, and others.

### DETAILED DESCRIPTION

The present invention seeks to provide medical device rental outlets (as opposed to medical clinics) for renting to users on a per-sessions basis electromedical devices intended for multi-sessions treatments of various indications at the outlet or close to it. As opposed to medical clinics, such outlets are merely rental outlets, and as such, are subject to less regulations, are easier to manage, and are less expensive to set up and run.

Presently, there are medical devices that are rented to users of home healthcare equipment, such as beds, wheelchairs, inhalators, electrical muscle stimulators, and many more. Other energy-emitting electromedical devices configured and regulated for home-use may be used for treatment of pain reduction, erectile dysfunction, diabetic foot ulcers, and many more. Such energy-emitting treatments, called sequential treatments, may include multiple sessions of less than one hour each, with inter-session breaks of several days each.

Sequential treatments performed as home treatments have several disadvantages. For the lessee (home user) of the equipment, renting or leasing a device for the treatment duration can be very expensive due to the relatively long time duration of the treatment. In other words, although each session may be short, the whole treatment extends over a long period of time. Thus, the rental/leasing fee for the duration of the treatment is prohibitively high.

For the lessor (owner or licensee) of the equipment, sequential treatments performed as home treatments have a low rental throughput (low number of treatments per unit time), and there is a relatively high and expensive risk of device damage during treatment and transportation.

Alternatively, setting up a mobile operation, in which the medical devices may be moved from patient to patient, may increase the throughput of home treatments per device, but the rental cost increases due to the device transportation, regulatory requirements for performing treatment and added risk of damage. Moreover, operating several devices in parallel requires an operator for each device.

Thus, a low cost high throughput system is desired for sequential treatments.

The invention solves these problems by providing a network system for affordable rental of medical devices configured for home use. Since home-use devices can also be used outside the user's home, the invention provides a leasing/rental network of outlets for renting such home-use devices to be self-operated by users (as opposed to by medical staff) at or geographically close to the outlet. Since multiple users may perform sessions of sequential self-treatments in a common site using a common device, a high throughput and low operational cost may be achieved, in particular when a single trained operator simultaneously assists several self-treating users in operating respective devices.

Accordingly, each treatment is composed of sequentially-performed multiple sessions, which take place at a common site. The medical devices used in the system of the invention, include, without limitation, energy-emitting devices configured to deliver treating energy. In one aspect of the invention, no person other than the patient needs to contact the device during the delivery. Alternatively, the patient may be assisted, when needed, by one or more trained operators at the medical-device rental outlet. The devices used may be devices which have been configured for home treatment.

In one non-limiting example, the invention provides a site for users to rent energy-emitting devices for sequentially-performed multiple sessions for indications such as but not limited to, sexual wellness improvement (e.g., treatment of erectile dysfunction), or diabetic foot ulcer, and many others. One aspect of the invention is providing sequentially-performed multiple sessions using a low-intensity shockwaves device, which may be used for treating the area of the pelvis, peritoneum or penis, e.g., for treatment of erectile dysfunction, or other tissues and parts of the body, such as but not limited to, vaginal treatments, pain and cosmetic indications, and others.

The invention provides outlets that take advantage of the short sessions and alleviate the cost of long treatment by offering usage of an available device in an outlet for the duration of the session; there is no renting a particular device but sharing usage of available devices for self-treatments.

Sharing sites and devices increase devices usability and may reduce user expense for such a treatment. In addition, sessions can take place in various outlets, so there is no need to transport a device from place to place, in case of the patient traveling.

Thus, in one aspect of the invention, the outlets provide shared usage of devices by more than one self-treating patient where the treatments include multiple sessions that are performed over a period of time.

What is claimed is:

1. A method of treatment comprising:
providing one or more rental outlets for medical-devices, each of said outlets comprising one or more trained operators and one or more energy-emitting medical devices configured for home-use, and allowing or instructing a care recipient to rent said one or more energy-emitting medical devices to perform a multiplicity of sequentially-performed energy-emitting self-treatment sessions, assisted when needed by said one or more trained operators, which take place at said one or more rental outlets for medical-devices or at close proximity thereto.

2. The method according to claim 1, wherein said sequentially-performed energy-emitting treatment sessions are performed in more than one of said outlets.

3. The method according to claim 1, wherein said sequentially-performed energy-emitting treatment sessions comprise shared usage of said one or more energy-emitting medical devices for treatment of more than one care recipient.

4. The method according to claim 1, wherein said sequentially-performed energy-emitting treatment sessions are performed using a low-intensity shockwaves device.

5. The method according to claim 1, wherein said sequentially-performed energy-emitting treatment sessions are used for sexual wellness improvement.

6. The method according to claim 1, wherein said sequentially-performed energy-emitting treatment sessions are used for treatment of erectile dysfunction.

7. The method according to claim 1, wherein said sequentially-performed energy-emitting treatment sessions are used for treatment of a diabetic ulcer.

8. The method according to claim 1, wherein said sequentially-performed energy-emitting treatment sessions are used for treatment of a vagina.

9. The method according to claim 1, wherein said sequentially-performed energy-emitting treatment sessions are used for treatment of pain.

10. The method according to claim 1, wherein said sequentially-performed energy-emitting treatment sessions are used for a cosmetic treatment.

11. The method according to claim 1, wherein said care recipient pays a rental fee for each treatment session.
